(19) United States
(12) Patent Application Publication
(10) Pub. No.: US 2026/0338685 A1
(43) Pub. Date: Oct. 29, 2026

(54) BIOLOGICAL COMPONENT MEASUREMENT DEVICE AND BIOLOGICAL COMPONENT MEASUREMENT METHOD

(71) Applicant: Mitsubishi Electric Corporation, Tokyo (JP)

(72) Inventors: Keita MIYAGAWA, Chiyoda-ku, Tokyo (JP); Shusaku HAYASHI, Chiyoda-ku, Tokyo (JP); Yuki TSUDA, Chiyoda-ku, Tokyo (JP); Koichi AKIYAMA, Chiyoda-ku, Tokyo (JP)

(73) Assignee: Mitsubishi Electric Corporation, Tokyo (JP)

(21) Appl. No.: 19/486,391

(22) PCT Filed: Jun. 20, 2023
(86) PCT No.: PCT/JP2023/022786

§ 371 Date: Nov. 21, 2025

(51) Int. Cl. G01N 21/41; G01N 33/487

(52) U.S. Cl. CPC G01N 21/41; G01N 33/487; G01N 2201/061

(57) ABSTRACT

An excitation light source emits excitation light traveling through an optical medium toward a sample placed on a sample placement surface. A probe light source emits probe light traveling through the optical medium. A light position detector detects a position of emission probe light emitted from the optical medium. A computation unit calculates an amount or a concentration of a biological component in a biological substance, based on a position of the emission probe light detected by the light position detector when the sample is a referential substance, and a position of the emission probe light detected by the light position detector when the sample is the biological substance, wherein the referential substance absorbs the excitation light and has no specific absorption peak in an excitation wavelength of the excitation light.

### TECHNICAL FIELD

The present disclosure relates to a biological component measurement device and a biological component measurement method.

### BACKGROUND ART

Conventionally, biological component measurement devices and biological component measurement methods have been known. For example, a method described in Japanese National Patent Publication No. 2017-519214 (PTL 1) includes: disposing an optical medium on a surface of a substance to bring at least one region of a surface of the optical medium into contact with the surface of the substance; irradiating the surface of the substance with an excitation light flux having an excitation wavelength, through the region of the surface of the optical medium in contact with the surface of the substance; emitting a probe light flux, through the optical medium, onto the region of the surface of the optical medium in direct contact with the surface of the substance, such that the probe light flux overlaps the excitation light flux at an interface between the optical medium and the surface of the substance and the probe light flux is reflected at the interface; directly or indirectly detecting deflection of the reflected probe light flux according to the wavelength of the excitation light flux; and analyzing the substance based on the deflection of the reflected probe light flux depending on the wavelength of the excitation light flux.

### CITATION LIST

### Patent Literature

PTL 1: Japanese National Patent Publication No. 2017-519214

### SUMMARY OF INVENTION

### Technical Problem

In a device disclosed in PTL 1, an infrared light source is used to emit the excitation light flux. Since the infrared light source is influenced by heat accumulation inside the light source or an external factor such as a change in external air temperature, its output of infrared light is not stable. As a result, heat generation in a biological substance caused by irradiation of the infrared light also fluctuates, and thus accuracy of measuring the amount or concentration of a biological component is also not stable.

Accordingly, an object of the present disclosure is to provide a biological component measurement device and a biological component measurement method capable of accurately measuring the amount or concentration of a biological component without being influenced by instability of infrared light.

### Solution to Problem

A biological component measurement device in the present disclosure includes: an optical medium including a sample placement surface; an excitation light source to emit, toward a sample placed on the sample placement surface, excitation light traveling through the optical medium and having an excitation wavelength of the sample; a probe light source to emit probe light traveling through the optical medium; a light position detector to detect a position of emission probe light emitted from the optical medium; and a computation unit to calculate an amount or a concentration of a biological component in a biological substance, based on a position of the emission probe light detected by the light position detector when the sample is a referential substance, and a position of the emission probe light detected by the light position detector when the sample is the biological substance. The referential substance absorbs the excitation light and has no specific absorption peak in the excitation wavelength of the excitation light.

A biological component measurement method in the present disclosure includes: emitting, by an excitation light source, toward a sample placed on a sample placement surface of an optical medium, excitation light traveling through the optical medium and having an excitation wavelength of the sample; emitting, by a probe light source, probe light traveling through the optical medium; detecting, by a light position detector, a position of emission probe light of the probe light incident on the optical medium when the sample is a referential substance; detecting, by the light position detector, a position of emission probe light of the probe light incident on the optical medium when the sample is a biological substance; and calculating, by a computation unit, an amount or a concentration of a biological component in the biological substance, based on the position of the emission probe light detected by the light position detector when the sample is the referential substance, and the position of the emission probe light detected by the light position detector when the sample is the biological substance. The referential substance absorbs the excitation light and has no specific absorption peak in the excitation wavelength of the excitation light.

### Advantageous Effects of Invention

According to the present disclosure, since the amount or concentration of the biological component in the biological substance is calculated based on the position of the emission probe light detected by the light position detector when the sample is the referential substance and the position of the emission probe light detected by the light position detector when the sample is the biological substance, it is possible to accurately measure the amount or concentration of the biological component without being influenced by instability of infrared light.

### BRIEF DESCRIPTION OF DRAWINGS

FIG. 1 is a diagram illustrating a configuration of a biological component measurement device according to a first embodiment.

FIGS. 2(a) and 2(b) are diagrams each illustrating an optical path of probe light 7.

FIG. 3 is a flowchart illustrating a procedure of a biological component measurement method for the biological component measurement device in the first embodiment.

FIG. 4 is a diagram illustrating a configuration of a biological component analysis device in a modification of the first embodiment.

FIG. 5 is a flowchart illustrating a procedure of a biological component measurement method for a biological component measurement device in a second embodiment.

FIG. 6 is a diagram illustrating a configuration of a computation unit 11.

FIG. 7 is a diagram illustrating a configuration of a learning device 91 in a third embodiment.

FIG. 8 is a diagram illustrating a configuration of an inference device 95 in the third embodiment.

FIG. 9 is a diagram illustrating a configuration of a learning device 91A in a fourth embodiment.

FIG. 10 is a diagram illustrating a configuration of an inference device 95A in the fourth embodiment.

FIG. 11 is a diagram illustrating a configuration of a learning device 91B in a fifth embodiment.

FIG. 12 is a diagram illustrating a configuration of an inference device 95B in the fifth embodiment.

FIG. 13 is a diagram illustrating a configuration of a learning device 91C in a sixth embodiment.

FIG. 14 is a diagram illustrating a configuration of an inference device 95C in the sixth embodiment.

FIG. 15 is a diagram showing a configuration in a case where the function of computation unit 11 is implemented using software.

### DESCRIPTION OF EMBODIMENTS

### First Embodiment

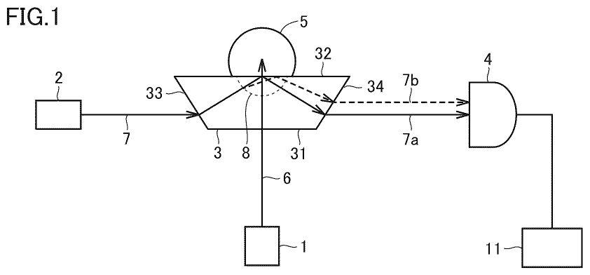

FIG. 1 is a diagram illustrating a configuration of a biological component measurement device in a first embodiment. The biological component measurement device includes an excitation light source 1, a probe light source 2, an optical medium 3, a light position detector 4, and a computation unit 11.

Excitation light source 1 includes at least one or more infrared light sources. Excitation light source 1 includes a broadband quantum cascade laser that emits infrared light in all or some of wavelength ranges including an excitation wavelength (a wavelength of a fingerprint spectrum) of a component to be measured in a sample 5. In a case where the component to be measured is sugar in a human body, it is assumed that wavelengths λ1, λ2, and λ3 are used for measurement, for example. Light having wavelength λ1 and light having wavelength λ2 are excitation wavelengths of the sugar in the human body, and are absorbed by the sugar in the human body. Light having wavelength λ3 is used as a referential wavelength that is not absorbed by the sugar in the human body and is absorbed by a referential substance. Four or more wavelengths may be used for measurement.

The infrared light emitted from excitation light source 1 is transmitted through optical medium 3 as excitation light 6, and is incident on sample 5. In the case of measuring a component contained in a living body, excitation light 6 is incident from the skin of a finger, an arm, an ear, or the like of the living body, to measure absorption of the light by a substance contained inside the living body, such as absorption by a substance contained in an interstitial fluid, for example.

Probe light source 2 emits probe light 7. Probe light 7 is incident on optical medium 3 from a third surface 33 of optical medium 3. Probe light 7 is refracted at third surface 33 and travels through optical medium 3 toward an interface between optical medium 3 (a second surface 32) and sample 5.

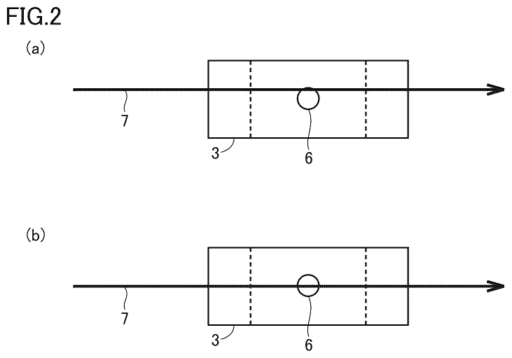

FIGS. 2(a) and 2(b) are diagrams each illustrating an optical path of probe light 7.

In plan view of a sample placement surface (second surface 32), the optical path of probe light 7 in optical medium 3 overlaps a portion of the sample placement surface (second surface 32) irradiated with excitation light 6, partially (FIG. 2(a)) or entirely (FIG. 2(b)).

Probe light 7 is totally internally reflected at the interface between optical medium 3 (second surface 32) and sample 5. While probe light 7 travels through optical medium 3, probe light 7 travels through a refractive index gradient region 8 generated in optical medium 3 by absorption heat of sample 5. Probe light 7 is refracted at refractive index gradient region 8, and a traveling direction of probe light 7 changes. Probe light 7 is emitted from a fourth surface 34 of optical medium 3.

The light outputted from probe light source 2 may have a wavelength in any wavelength band as long as the light is transmitted through optical medium 3. For example, it is possible to use a wavelength band such as a visible light region of 400 to 900 nm, which is mass-produced for many applications and inexpensive, or an invisible region having a wavelength band of 1300 to 1700 nm, which is used for optical fiber communication.

Optical medium 3 is made of a material through which excitation light 6 emitted from excitation light source 1 and incident probe light 7 emitted from probe light source 2 are transmitted. For example, optical medium 3 is made of a substance such as zinc sulfide (ZnS) or zinc selenide (ZnSe) that generally has a high transmittivity in a wavelength region of visible light to a wavelength region of infrared light. In a case where a measurement target is biological measurement, zinc sulfide (ZnS) can be used as optical medium 3 in consideration of biological stability. Chalcogenide glass having a thermal conductivity lower than that of zinc sulfide (ZnS) or zinc selenide (ZnSe) may be used such that a change in refractive index of optical medium 3 due to heat generated in sample 5 is localized.

A case where a light output of excitation light source 1 is zero (a reference state) will be described. Since the state inside optical medium 3 is uniform in the reference state, the light outputted from probe light source 2 is refracted only when it is incident on optical medium 3 and it is emitted from optical medium 3. A position at which emission probe light 7a is incident on light position detector 4 in the reference state is defined as a reference position RP. Although the present embodiment shows a path in which probe light 7a is totally reflected once at the interface between optical medium 3 and sample 5, as a path of probe light 7a, the path may be any path that passes through refractive index gradient region 8 generated in optical medium 3, and may be a path in which probe light 7a is totally reflected twice or more in the optical medium, or a path that passes through the vicinity of a contact surface between optical medium 3 and sample 5 in parallel to the contact surface.

Next, a case where there is an output from excitation light source 1 will be described. Excitation light source 1 outputs the infrared light having the fingerprint spectrum wavelength of the component to be measured in sample 5, as excitation light 6. Excitation light 6 is incident on sample 5 through optical medium 3. Excitation light 6 is absorbed by sample 5. Absorption heat generated by absorption is generated in sample 5. The generated absorption heat propagates into optical medium 3, and a temperature gradient is generated inside optical medium 3. Since the refractive index of the optical medium generally has temperature dependency, refractive index gradient region 8 is formed according to the temperature gradient. This state is defined as a changed state.

In the changed state, incident probe light 7 outputted from probe light source 2 passes through refractive index gradient region 8. Incident probe light 7 is refracted according to a slope of the refractive index at a position where it passes through refractive index gradient region 8. Refracted incident probe light 7 is emitted from optical medium 3 as emission probe light 7b, and is incident on light position detector 4. A position at which emission probe light 7b is incident on light position detector 4 in the changed state is defined as CP. Although FIG. 1 shows the incident positions in a height direction only, the incident positions are also displaced in a horizontal direction. Incident positions of excitation light 6 and incident probe light 7 are adjusted such that a difference between reference position RP and position CP becomes maximum, that is, a refractive index gradient becomes maximum.

When the component to be measured in sample 5 (that is, a component having a high absorption coefficient at a certain wavelength of the light emitted from excitation light source 1) has a large amount, the amount of light absorption in sample 5 increases, and thus the amount of generated heat increases. As a result, the refractive index gradient increases. The relation between the amount of the component to be measured in sample 5 and the difference between reference position RP at which emission probe light 7a is incident on light position detector 4 in the reference state and position CP at which emission probe light 7b is incident on light position detector 4 in the changed state is substantially proportional.

Excitation light source 1 essentially has an output determined for each wavelength.

However, excitation light source 1 is influenced by heat accumulation inside excitation light source 1 and an external factor such as a change in external air temperature. In measurement of a biological substance, influence of an external factor is included in position CP of emission probe light 7b. In the present embodiment, an output reflecting the influence of an external factor is measured by absorption spectroscopy using a referential substance. Since the absorption spectroscopy uses ease of light absorption by a substance for the light having that wavelength (an absorption coefficient), a substance having a constant absorption coefficient (no specific absorption peak) in a wavelength band of excitation light source 1 is appropriate as the referential substance. Measurement using such a referential substance as sample 5 is equivalent to measurement of an output of the excitation light according to the absorption coefficient of that substance.

In the present embodiment, the influence of an external factor is removed using not only a position CP(B) of emission probe light 7b(B) when the biological substance is used as sample 5 but also a position CP(A) of emission probe light 7b(A) when the referential substance is used as sample 5. Position CP(A) serves as an evaluation indicator of stability of a light output of excitation light 6 at the time of measurement. By detecting position CP(A) when sample 5 is the referential substance before measuring the amount or concentration of a biological component in the biological substance, it is possible to perform measurement while removing the influence of an external factor on excitation light source 1. As the referential substance, a substance which absorbs the wavelength band of excitation light source 1, has no specific absorption peak in that wavelength band, and comes into close contact with optical medium 3 is selected. For example, in the case of an infrared light region, water or the like can be used. A refractive index gradient formed when the referential substance is sample 5 is basically different from a refractive index gradient formed when the biological substance is sample 5.

Computation unit 11 computes an output from light position detector 4. Computation unit 11 measures the amount or concentration of a biological component (such as a sugar content) in the biological substance, based on position CP(A) of emission probe light 7b detected by light position detector 4 when sample 5 is the referential substance, and position CP(B) of emission probe light 7b detected by light position detector 4 when sample 5 is the biological substance. For example, computation unit 11 calculates CP(B)-CP(A) or CP(B)/CP(A), to obtain the amount or concentration of the component in the biological substance (for example, the sugar content in the skin).

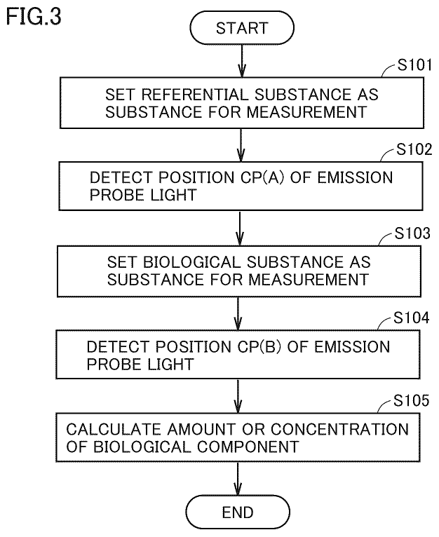

FIG. 3 is a flowchart illustrating a procedure of a biological component measurement method for the biological component measurement device in the first embodiment.

In step S101, the referential substance (for example, water) is set as sample 5.

In step S102, in the changed state in which excitation light source 1 irradiates sample 5 with excitation light 6, light position detector 4 detects position CP(A) of emission probe light 7b(A).

In step S103, the biological substance (for example, skin) is set as sample 5.

In step S104, in the changed state in which excitation light source 1 irradiates sample 5 with excitation light 6, light position detector 4 detects position CP(B) of emission probe light 7b(B).

In step S105, computation unit 11 calculates the amount or concentration of the component in the biological substance (for example, the sugar content in the skin), based on position CP(A) and position CP(B). For example, computation unit 11 calculates CP(B)-CP(A) or CP(B)/CP(A), to obtain the amount or concentration of the component in the biological substance (for example, the sugar content in the skin).

According to the present embodiment, by using the result of measurement when the referential substance is used as sample 5, the output of the excitation light influenced by an external factor can be monitored without adding a new constituent element. Since instability of the excitation light can be removed by performing the measurement of the referential substance and the measurement of the biological substance as a set, the amount or concentration of the biological component can be accurately measured.

It should be noted that a difference between position CP(A) and reference position RP may be used instead of position CP(A), and a difference between position CP(B) and reference position RP may be used instead of position CP(B).

### Modification of First Embodiment

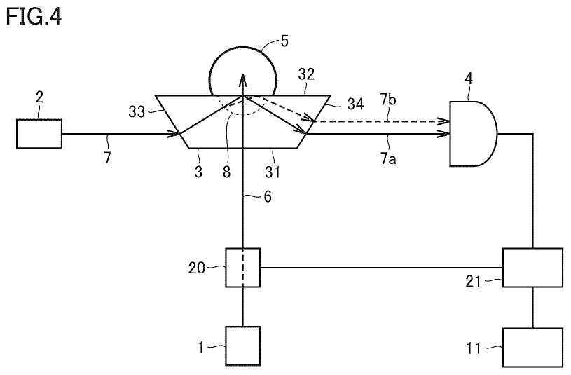

FIG. 4 is a diagram illustrating a configuration of a biological component analysis device in a modification of the first embodiment. The biological component analysis device in the modification of the first embodiment is different from the non-invasive component analysis device in the first embodiment in that the biological component analysis device in the modification of the first embodiment includes an optical chopper 20 between excitation light source 1 and optical medium 3, and a lock-in amplifier 21 between light position detector 4 and computation unit 11.

Optical chopper 20 is disposed in an optical path of excitation light 6. Optical chopper 20 chops excitation light 6 (continuous light) emitted from excitation light source 1 at an arbitrary frequency. Excitation light 6 becomes intermittent light (pulsed light) that is turned on and off at a cycle corresponding to a chopping frequency of optical chopper 20 (a frequency at which the light is turned on and off), and is incident on optical medium 3. A well-known configuration can be applied to optical chopper 20. For example, optical chopper 20 has a rotating disk in which an opened portion that allows passage of excitation light 6 and a light blocking portion that blocks excitation light 6 are disposed in a circumferential direction, and a motor that rotates the rotating disk. Whether or not to irradiate sample 5 with excitation light 6 can be switched by cyclically rotating the rotating disk by the motor. That is, excitation light 6 undergoes intensity modulation at the chopping frequency of optical chopper 20. A chopping frequency of excitation light 6 is determined by a rotation speed of the rotating disk.

Optical chopper 20 and lock-in amplifier 21 are connected to an oscillator (not shown). The oscillator sets the chopping frequency (a modulation frequency) of optical chopper 20. The oscillator generates a control signal for chopper control of excitation light 6, and supplies the generated control signal to optical chopper 20 and lock-in amplifier 21. The control signal includes the chopping frequency of optical chopper 20.

Lock-in amplifier 21 selectively amplifies a signal synchronized with the chopping frequency (modulation frequency) of optical chopper 20, among signals related to the position of probe light 7 outputted from light position detector 4. An ON period of a chopping cycle corresponds to a period in which excitation light 6 is emitted. An OFF period of the chopping cycle corresponds to a period in which excitation light 6 is not emitted.

### Second Embodiment

A case where a biological substance for measurement is composed of a plurality of biological components and the amount or concentration of one of the biological components is to be measured will be considered. For example, when the biological substance is human skin, excitation light 6 is absorbed by a plurality of components such as a sugar content, an oil content, and sweat components in the skin, and it is difficult to measure the amount or concentration of one biological component just by measurement of the skin.

A reference substance which absorbs the wavelength band of excitation light source 1, has an absorption peak(s) of one or more biological components in that wavelength band, and comes into close contact with optical medium 3 is selected. For example, a sebum deposit or sweat itself on the surface of a living body can be used as the reference substance. By using the result of measurement when the reference substance is used as sample 5, it is possible to remove contribution of a biological component other than the biological component to be measured. A refractive index gradient formed when the reference substance is sample 5 is basically different from the refractive index gradient formed when the biological substance is sample 5.

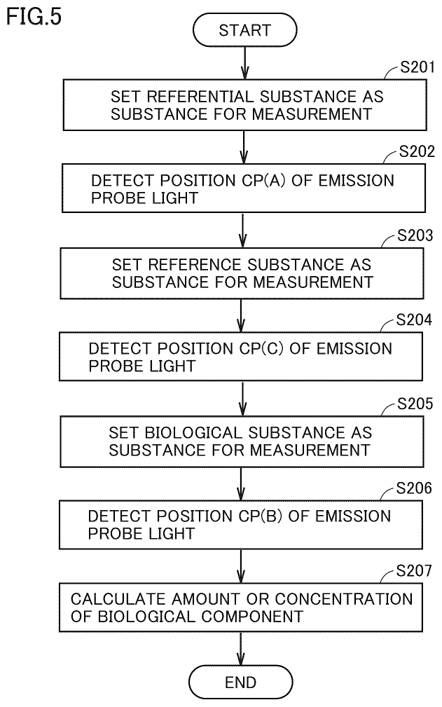

FIG. 5 is a flowchart illustrating a procedure of a biological component measurement method for a biological component measurement device in a second embodiment.

In step S201, a referential substance (for example, water) is set as sample 5.

In step S202, in the changed state in which excitation light source 1 irradiates sample 5 with excitation light 6, light position detector 4 detects position CP(A) of emission probe light 7b(A).

In step S203, the reference substance (for example, a sebum deposit or sweat on the surface of the living body) is set as sample 5.

In step S204, in the changed state in which excitation light source 1 irradiates sample 5 with excitation light 6, light position detector 4 detects a position CP(C) of emission probe light 7b(C).

In step S205, the biological substance (for example, skin) is set as sample 5.

In step S206, in the changed state in which excitation light source 1 irradiates sample 5 with excitation light 6, light position detector 4 detects position CP(B) of emission probe light 7b(B).

In step S207, computation unit 11 calculates the amount or concentration of a component in the biological substance (for example, a sugar content in the skin), based on position CP(A), position CP(B), and position CP(C). For example, computation unit 11 calculates CP(B)-CP(A)-CP(C), to obtain the amount or concentration of the component in the biological substance (for example, the sugar content in the skin).

It should be noted that the difference between position CP(A) and reference position RP may be used instead of position CP(A), the difference between position CP(B) and reference position RP may be used instead of position CP(B), and a difference between position CP(C) and reference position RP may be used instead of position CP(C).

### Third Embodiment

The first embodiment has described that, by performing the measurement when the referential substance is sample 5 and the measurement when the biological substance is sample 5 as a set, the amount or concentration of the biological component in the biological substance can be measured while removing the influence of an external factor on excitation light source 1. In the present embodiment, detected CP(A) and CP(B) are used to calculate the amount or concentration of a component in a biological substance (for example, a sugar content in the skin) with higher accuracy.

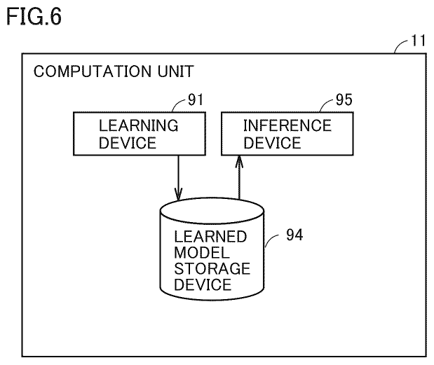

FIG. 6 is a diagram illustrating a configuration of computation unit 11.

Computation unit 11 includes a learning device 91, an inference device 95, and a learned model storage device 94.

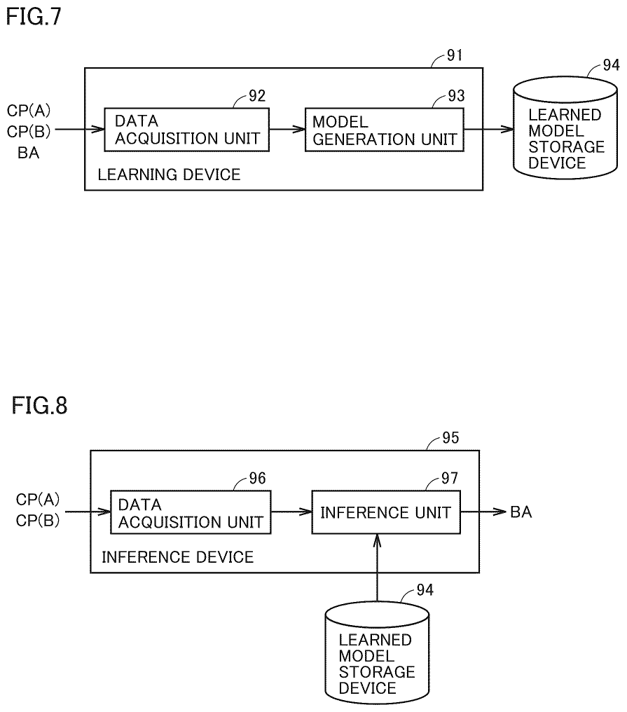

FIG. 7 is a diagram illustrating a configuration of learning device 91 in a third embodiment.

Learning device 91 includes a data acquisition unit 92 and a model generation unit 93.

Data acquisition unit 92 acquires learning data including position CP(A) of emission probe light 7b(A) detected by light position detector 4 when sample 5 is a referential substance, position CP(B) of emission probe light 7b(A) detected by light position detector 4 when sample 5 is the biological substance, and an amount or concentration BA of a biological component. The learning data is data in which position CP(A) of emission probe light 7b(A) and position CP(B) of emission probe light 7b(A) are associated with amount or concentration (a correct answer) BA of the biological component.

Using the learning data, model generation unit 93 generates a learned model for inferring amount or concentration BA of the biological component, from position CP(A) of emission probe light 7b(A) detected by light position detector 4 when sample 5 is the referential substance, and position CP(B) of emission probe light 7b(B) detected by light position detector 4 when sample 5 is the biological substance.

Learning device 91 and inference device 95 are not limited to those embedded in computation unit 11. For example, learning device 91 and inference device 95 may be those connected to the biological component measurement device via a network. Learning device 91 and inference device 95 may exist on a cloud server.

As a learning algorithm used by model generation unit 93, a known algorithm such as supervised learning, unsupervised learning, or reinforcement learning can be used. As an example, a case where a neural network is applied will be described.

Model generation unit 93 performs so-called supervised learning according to a neural network model, for example. Here, supervised learning is a technique of providing the learning device with a set of data of an input and a result (label) to learn a feature in the learning data, and inferring the result from the input. The neural network is constituted by an input layer including a plurality of neurons, an intermediate layer (a hidden layer) including a plurality of neurons, and an output layer including a plurality of neurons. The intermediate layer may be one layer, or two or more layers. For example, in the case of a three-layer neural network, when a plurality of inputs are inputted to the input layer, values thereof are multiplied by a weight W1 and are inputted to the intermediate layer, results thereof are further multiplied by a weight W2, and a result is outputted from the output layer. This outputted result varies depending on values of weights W1 and W2. The neural network performs so-called supervised learning according to the learning data acquired by data acquisition unit 92. That is, the neural network learns by adjusting weights W1 and W2 such that the result outputted from the output layer, as a result of inputting position CP(A) of emission probe light 7b(A) detected by light position detector 4 when sample 5 is the referential substance and position CP(B) of emission probe light 7b(B) detected by light position detector 4 when sample 5 is the biological substance to the input layer, is close to amount or concentration BA (correct answer) of the biological component.

Model generation unit 93 generates the learned model by performing learning as described above, and outputs it.

Learned model storage device 94 stores the learned model outputted from model generation unit 93.

FIG. 8 is a diagram illustrating a configuration of inference device 95 in the third embodiment. Inference device 95 includes a data acquisition unit 96 and an inference unit 97.

Data acquisition unit 96 acquires position CP(A) of emission probe light 7b(A) detected by light position detector 4 when sample 5 is the referential substance, and position CP(B) of emission probe light 7b(B) detected by light position detector 4 when sample 5 is the biological substance.

Inference unit 97 infers amount or concentration BA of the biological component, from position CP(A) of emission probe light 7b(A) detected by light position detector 4 when sample 5 is the referential substance and position CP(B) of emission probe light 7b(B) detected by light position detector 4 when sample 5 is the biological substance, the positions being acquired by data acquisition unit 96, using the learned model stored in learned model storage device 94 for inferring amount or concentration BA of the biological component from position CP(A) of emission probe light 7b(A) detected by light position detector 4 when sample 5 is the referential substance and position CP(B) of emission probe light 7b(B) detected by light position detector 4 when sample 5 is the biological substance.

Although inference unit 97 uses the learned model learned by model generation unit 93 in the present embodiment, it may use a learned model generated outside the biological component measurement device.

Although the present embodiment has described a case where supervised learning is applied as a learning algorithm used by model generation unit 93, the present disclosure is not limited thereto. As a learning algorithm, reinforcement learning, unsupervised learning, semi-supervised learning, or the like can be applied, instead of supervised learning.

As a learning algorithm used by model generation unit 93, deep learning that learns extraction of a feature amount itself can also be used, or machine learning may be performed according to other known methods such as genetic programming, functional logic programming, and a support vector machine, for example.

According to the present embodiment, the amount or concentration of the biological component to be measured can be measured with high accuracy,

### Fourth Embodiment

In the present embodiment, computation unit 11 includes a learning device 91A, an inference device 95A, and a learned model storage device 94A.

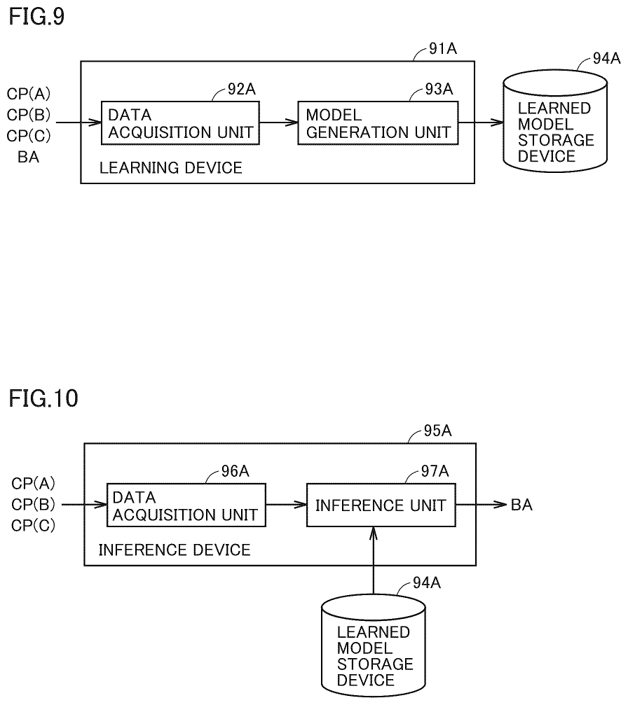

FIG. 9 is a diagram illustrating a configuration of learning device 91A in a fourth embodiment. Learning device 91A includes a data acquisition unit 92A and a model generation unit 93A.

Data acquisition unit 92A acquires learning data including position CP(A) of emission probe light 7b(A) detected by light position detector 4 when sample 5 is a referential substance, position CP(C) of emission probe light 7b(C) detected by light position detector 4 when sample 5 is a reference substance, position CP(B) of emission probe light 7b(B) detected by light position detector 4 when sample 5 is a biological substance, and amount or concentration BA of a biological component.

Using the learning data, model generation unit 93A generates a learned model for inferring amount or concentration BA of the biological component, from position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when sample 5 is the biological substance.

Learned model storage device 94A stores the learned model outputted from model generation unit 93A.

FIG. 10 is a diagram illustrating a configuration of inference device 95A in the fourth embodiment. Inference device 95A includes a data acquisition unit 96A and an inference unit 97A.

Data acquisition unit 96A acquires position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when the sample 5 is the biological substance.

Inference unit 97A infers amount or concentration BA of the biological component, from position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when sample 5 is the biological substance, the positions being acquired by data acquisition unit 96A, using the learned model stored in learned model storage device 94A for inferring amount or concentration BA of the biological component from position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when sample 5 is the biological substance.

According to the present embodiment, the amount or concentration of the biological component to be measured can be measured with high accuracy.

### Fifth Embodiment

Computation unit 11 according to a fifth embodiment includes a learning device 91B, an inference device 95B, and a learned model storage device 94B.

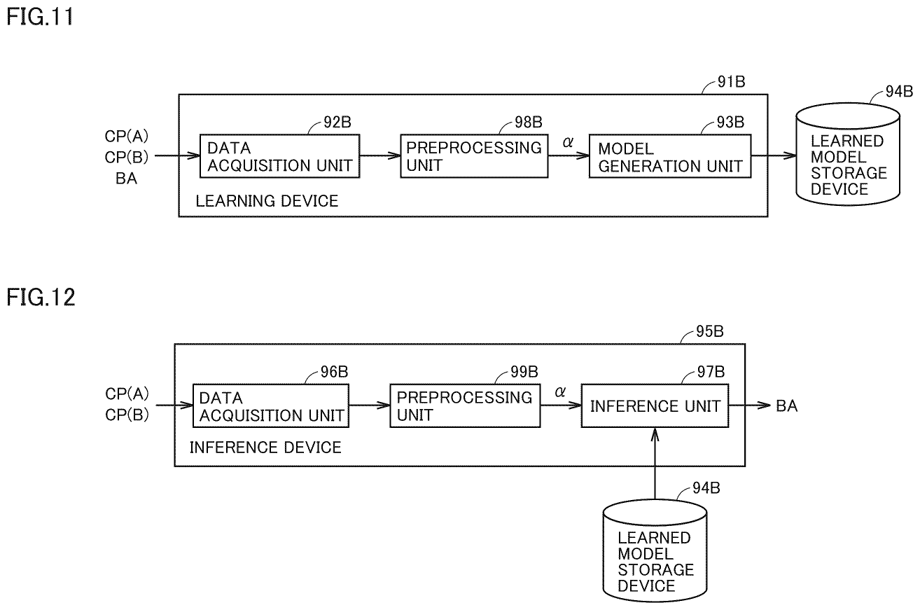

FIG. 11 is a diagram illustrating a configuration of learning device 91B in the fifth embodiment. Learning device 91B includes a data acquisition unit 92B, a preprocessing unit 98B, and a model generation unit 93B.

Data acquisition unit 92B acquires data including position CP(A) of emission probe light 7b(A) detected by light position detector 4 when sample 5 is a referential substance, position CP(B) of emission probe light 7b(B) detected by light position detector 4 when sample 5 is a biological substance, and amount or concentration BA of a biological component.

Preprocessing unit 98B calculates one indicator a, from position CP(A) of emission probe light 7b(A) detected by light position detector 4 when sample 5 is the referential substance, and position CP(B) of emission probe light 7b(B) detected by light position detector 4 when sample 5 is the biological substance. Indicator α is data in which the biological component is manifested. Indicator α can be, for example, CP(B)-CP(A) or CP(B)/CP(A). By preprocessing unit 98B, it is possible to reduce the amount of data and time related to learning, and to avoid overlearning.

Model generation unit 93B generates a learned model for inferring amount or concentration BA of the biological component from indicator α, using learning data including indicator α and amount or concentration BA of the biological component.

Learned model storage device 94B stores the learned model outputted from model generation unit 93B.

FIG. 12 is a diagram illustrating a configuration of inference device 95B in the fifth embodiment. Inference device 95B includes a data acquisition unit 96B, a preprocessing unit 99B, and an inference unit 97B.

Data acquisition unit 96B acquires position CP(A) of emission probe light 7b(A) detected by light position detector 4 when sample 5 is the referential substance, and position CP(B) of emission probe light 7b(B) detected by light position detector 4 when sample 5 is the biological substance.

Preprocessing unit 99B calculates one indicator α, from position CP(A) of emission probe light 7b(A) detected by light position detector 4 when sample 5 is the referential substance, and position CP(B) of emission probe light 7b(B) detected by light position detector 4 when sample 5 is the biological substance.

Inference unit 97B infers amount or concentration BA of the biological component from indicator α calculated by preprocessing unit 99B, using the learned model stored in learned model storage device 94B for inferring amount or concentration BA of the biological component from indicator α.

According to the present embodiment, it is possible to reduce the amount of data and time related to learning, and to measure the amount or concentration of the biological component to be measured with high accuracy and with ease.

### Sixth Embodiment

Computation unit 11 in a sixth embodiment includes a learning device 91C, an inference device 95C, and a learned model storage device 94C.

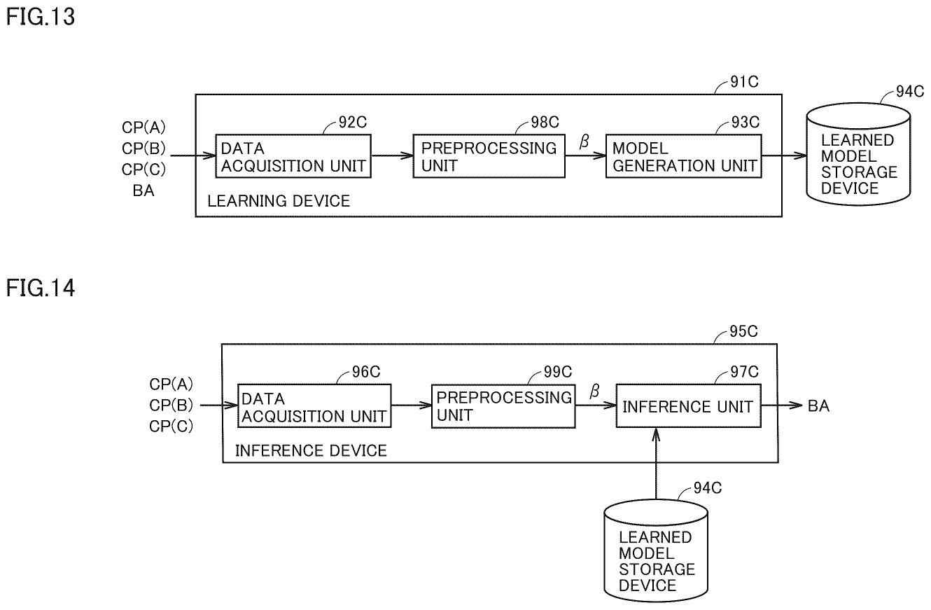

FIG. 13 is a diagram illustrating a configuration of learning device 91C in the sixth embodiment. Learning device 91C includes a data acquisition unit 92C, a preprocessing unit 98C, and a model generation unit 93C.

Data acquisition unit 92C acquires data including position CP(A) of emission probe light 7b(A) detected by light position detector 4 when sample 5 is a referential substance, position CP(C) of emission probe light 7b(C) detected by light position detector 4 when sample 5 is a reference substance, position CP(B) of emission probe light 7b(B) detected by light position detector 4 when sample 5 is a biological substance, and the amount or concentration of a biological component.

Preprocessing unit 98C calculates one indicator β, from position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when sample 5 is the biological substance.

Model generation unit 93C generates a learned model for inferring amount or concentration BA of the biological component from indicator β, using learning data including indicator β and amount or concentration BA of the biological component.

Learned model storage device 94C stores the learned model outputted from model generation unit 93C.

FIG. 14 is a diagram illustrating a configuration of inference device 95C in the sixth embodiment. Inference device 95C includes a data acquisition unit 96C, a preprocessing unit 99C, and an inference unit 97C.

Data acquisition unit 96C acquires position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when sample 5 is the biological substance.

Preprocessing unit 99C calculates one indicator β, from position CP(A) of emission probe light 7b(A) detected by light position detector 4 when sample 5 is the referential substance, position CP(C) of emission probe light 7b(C) detected by light position detector 4 when sample 5 is the reference substance, and position CP(B) of emission probe light 7b(B) detected by light position detector 4 when sample 5 is the biological substance. Indicator β can be, for example, CP(B)-CP(A)-CP(C).

Inference unit 97C infers amount or concentration BA of the biological component from indicator β calculated by preprocessing unit 99C, using the learned model stored in learned model storage device 94C for inferring amount or concentration BA of the biological component from indicator β.

According to the present embodiment, it is possible to reduce the amount of data and time related to learning, and to measure the amount or concentration of the biological component to be measured with high accuracy and with ease.

In each of the first to sixth embodiments described above, a corresponding operation of computation unit 11 may be configured by hardware or software of a digital circuit.

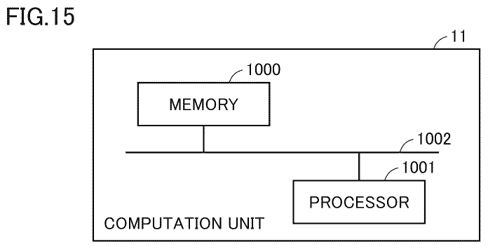

FIG. 15 is a diagram showing a configuration in a case where the function of computation unit 11 is implemented using software. Computation unit 11 includes a processor 1001 and a memory 1000 which are connected to a bus 1002. Processor 1001 executes a program stored in memory 1000.

It should be understood that the embodiments disclosed herein are illustrative and non-restrictive in every respect. The scope of the present disclosure is defined by the scope of the claims, rather than the description above, and is intended to include any modifications within the scope and meaning equivalent to the scope of the claims.

### REFERENCE SIGNS LIST

1: excitation light source; 2: probe light source; 3: optical medium; 4: light position detector; 5: sample; 6: excitation light; 7, 7a, 7b: probe light; 8: refractive index gradient region; 11: computation unit; 20: optical chopper; 21: lock-in amplifier; 91, 91A, 91B, 91C: learning device; 92, 92A, 92B, 92C, 96, 96A, 96B, 96C: data acquisition unit; 93, 93A, 93B, 93C: model generation unit; 94, 94A, 94B, 94C: learned model storage device; 95, 95A, 95B, 95C: inference device; 97A, 97B, 97C: inference unit; 98B, 98C, 99B, 99C: preprocessing unit; 1000: memory; 1001: processor; 1002: bus.

1. A biological component measurement device to measure an amount or a concentration of a sugar component in a biological substance, the biological component measurement device comprising:
an optical medium including a sample placement surface;
an excitation light source to emit, toward a sample placed on the sample placement surface, excitation light traveling through the optical medium, exciting the sugar component, and having an excitation wavelength that is absorbed by the sugar component and a referential wavelength that is not absorbed by the sugar component and is absorbed by a referential substance;
a probe light source to emit probe light traveling through the optical medium;
a light position detector to detect a position of emission probe light emitted from the optical medium; and
a computation circuitry to calculate the amount or concentration of the sugar component in the biological substance, based on a position of the emission probe light detected by the light position detector when the sample is the referential substance, and a position of the emission probe light detected by the light position detector when the sample is the biological substance, using the excitation light exciting the sugar component, and having the excitation wavelength that is absorbed by the sugar component and the referential wavelength that is not absorbed by the sugar component and is absorbed by the referential substance, the excitation light being emitted by the excitation light source, the referential substance absorbing the excitation light and having no specific absorption peak in the excitation wavelength of the excitation light, wherein
the computation circuitry further calculates the amount or concentration of the sugar component, based on a position of the emission probe light detected by the light position detector when the sample is a reference substance, the reference substance absorbing the excitation light and having an absorption peak(s) of one or more sugar components in the excitation wavelength of the excitation light.

2.-6. (canceled)

7. The biological component measurement device according to claim 1, wherein
the computation circuitry includes a learning device, and the learning device includes
a data acquisition circuitry to acquire learning data including the position of the emission probe light detected by the light position detector when the sample is the referential substance, the position of the emission probe light detected by the light position detector when the sample is the reference substance, the position of the emission probe light detected by the light position detector when the sample is the biological substance, and the amount or concentration of the sugar component, and
a model generation circuitry to generate a learned model for inferring the amount or concentration of the sugar component, from the position of the emission probe light detected by the light position detector when the sample is the referential substance, the position of the emission probe light detected by the light position detector when the sample is the reference substance, and the position of the emission probe light detected by the light position detector when the sample is the biological substance, using the learning data.

8. The biological component measurement device according to claim 1, wherein
the computation circuitry includes a learning device, and the learning device includes
a data acquisition circuitry to acquire data including the position of the emission probe light detected by the light position detector when the sample is the referential substance, the position of the emission probe light detected by the light position detector when the sample is the reference substance, the position of the emission probe light detected by the light position detector when the sample is the biological substance, and the amount or concentration of the sugar component,
a preprocessing circuitry to calculate one indicator, from the position of the emission probe light detected by the light position detector when the sample is the referential substance, the position of the emission probe light detected by the light position detector when the sample is the reference substance, and the position of the emission probe light detected by the light position detector when the sample is the biological substance, and
a model generation circuitry to generate a learned model for inferring the amount or concentration of the sugar component from the indicator, using learning data including the indicator and the amount or concentration of the sugar component.

9. The biological component measurement device according to claim 1, wherein
the computation circuitry includes an inference device, and the inference device includes
a data acquisition circuitry to acquire the position of the emission probe light detected by the light position detector when the sample is the referential substance, the position of the emission probe light detected by the light position detector when the sample is the reference substance, and the position of the emission probe light detected by the light position detector when the sample is the biological substance, and
an inference circuitry to infer the amount or concentration of the sugar component, from the position of the emission probe light detected by the light position detector when the sample is the referential substance, the position of the emission probe light detected by the light position detector when the sample is the reference substance, and the position of the emission probe light detected by the light position detector when the sample is the biological substance, the positions being acquired by the data acquisition circuitry, using a learned model for inferring the amount or concentration of the sugar component from the position of the emission probe light detected by the light position detector when the sample is the referential substance, the position of the emission probe light detected by the light position detector when the sample is the reference substance, and the position of the emission probe light detected by the light position detector when the sample is the biological substance.

10. The biological component measurement device according to claim 1, wherein
the computation circuitry includes an inference device, and the inference device includes
a data acquisition circuitry to acquire the position of the emission probe light detected by the light position detector when the sample is the referential substance, the position of the emission probe light detected by the light position detector when the sample is the reference substance, and the position of the emission probe light detected by the light position detector when the sample is the biological substance,
a preprocessing circuitry to calculate one indicator, from the position of the emission probe light detected by the light position detector when the sample is the referential substance, the position of the emission probe light detected by the light position detector when the sample is the reference substance, and the position of the emission probe light detected by the light position detector when the sample is the biological substance, and
an inference circuitry to infer the amount or concentration of the sugar component from the indicator calculated by the preprocessing circuitry, using a learned model for inferring the amount or concentration of the sugar component from the indicator.

11. The biological component measurement device according to claim 1, wherein, in plan view of the sample placement surface, an optical path of the probe light in the optical medium overlaps a portion of the sample placement surface irradiated with the excitation light.

12. A biological component measurement method to measure an amount or a concentration of a sugar component in a biological substance, the method comprising:
emitting, by an excitation light source, toward a sample placed on a sample placement surface of an optical medium, excitation light traveling through the optical medium, exciting the sugar component, and having an excitation wavelength that is absorbed by the sugar component and a referential wavelength that is not absorbed by the sugar component and is absorbed by a referential substance;
emitting, by a probe light source, probe light traveling through the optical medium;
detecting, by a light position detector, a position of emission probe light of the probe light incident on the optical medium when the sample is the referential substance;
detecting, by the light position detector, a position of emission probe light of the probe light incident on the optical medium when the sample is the biological substance;
calculating, by a computation circuitry, the amount or concentration of the sugar component in the biological substance, based on the position of the emission probe light detected by the light position detector when the sample is the referential substance, and the position of the emission probe light detected by the light position detector when the sample is the biological substance, using the excitation light exciting the sugar component, and having the excitation wavelength that is absorbed by the sugar component and the referential wavelength that is not absorbed by the sugar component and is absorbed by the referential substance, the excitation light being emitted by the excitation light source, the referential substance absorbing the excitation light and having no specific absorption peak in the excitation wavelength of the excitation light; and
detecting, by the light position detector, a position of emission probe light of the probe light incident on the optical medium when the sample is a reference substance, wherein
calculating the amount or concentration of the sugar component includes
calculating, by the computation circuitry, the amount or concentration of the sugar component, based on the position of the emission probe light detected by the light position detector when the sample is the referential substance, the position of the emission probe light detected by the light position detector when the sample is the reference substance, and the position of the emission probe light detected by the light position detector when the sample is the biological substance,
the reference substance absorbing the excitation light and having an absorption peak(s) of one or more sugar components in the excitation wavelength of the excitation light.

13. (canceled)

14. A biological component measurement device comprising:
an optical medium including a sample placement surface;
an excitation light source to emit, toward a sample placed on the sample placement surface, excitation light traveling through the optical medium and having an excitation wavelength of the sample;
a probe light source to emit probe light traveling through the optical medium;
a light position detector to detect a position of emission probe light emitted from the optical medium; and
a computation circuitry to calculate an amount or a concentration of a sugar component in a biological substance, based on a position of the emission probe light detected by the light position detector when the sample is a referential substance, and a position of the emission probe light detected by the light position detector when the sample is the biological substance, the referential substance absorbing the excitation light and having no specific absorption peak in the excitation wavelength of the excitation light, wherein
the computation circuitry further calculates the amount or concentration of the sugar component, based on a position of the emission probe light detected by the light position detector when the sample is a reference substance, the reference substance absorbing the excitation light and having an absorption peak(s) of one or more sugar components in the excitation wavelength of the excitation light, and
the computation circuitry includes a learning device, and the learning device includes
a data acquisition circuitry to acquire learning data including the position of the emission probe light detected by the light position detector when the sample is the referential substance, the position of the emission probe light detected by the light position detector when the sample is the reference substance, the position of the emission probe light detected by the light position detector when the sample is the biological substance, and the amount or concentration of the sugar component, and
a model generation circuitry to generate a learned model for inferring the amount or concentration of the sugar component, from the position of the emission probe light detected by the light position detector when the sample is the referential substance, the position of the emission probe light detected by the light position detector when the sample is the reference substance, and the position of the emission probe light detected by the light position detector when the sample is the biological substance, using the learning data.

15. A biological component measurement device comprising:
an optical medium including a sample placement surface;
an excitation light source to emit, toward a sample placed on the sample placement surface, excitation light traveling through the optical medium and having an excitation wavelength of the sample;
a probe light source to emit probe light traveling through the optical medium;
a light position detector to detect a position of emission probe light emitted from the optical medium; and
a computation circuitry to calculate an amount or a concentration of a sugar component in a biological substance, based on a position of the emission probe light detected by the light position detector when the sample is a referential substance, and a position of the emission probe light detected by the light position detector when the sample is the biological substance, the referential substance absorbing the excitation light and having no specific absorption peak in the excitation wavelength of the excitation light, wherein
the computation circuitry further calculates the amount or concentration of the sugar component, based on a position of the emission probe light detected by the light position detector when the sample is a reference substance, the reference substance absorbing the excitation light and having an absorption peak(s) of one or more sugar components in the excitation wavelength of the excitation light, and
the computation circuitry includes a learning device, and the learning device includes
a data acquisition circuitry to acquire data including the position of the emission probe light detected by the light position detector when the sample is the referential substance, the position of the emission probe light detected by the light position detector when the sample is the reference substance, the position of the emission probe light detected by the light position detector when the sample is the biological substance, and the amount or concentration of the sugar component,
a preprocessing circuitry to calculate one indicator, from the position of the emission probe light detected by the light position detector when the sample is the referential substance, the position of the emission probe light detected by the light position detector when the sample is the reference substance, and the position of the emission probe light detected by the light position detector when the sample is the biological substance, and
a model generation circuitry to generate a learned model for inferring the amount or concentration of the sugar component from the indicator, using learning data including the indicator and the amount or concentration of the sugar component.

16. A biological component measurement device comprising:
an optical medium including a sample placement surface;
an excitation light source to emit, toward a sample placed on the sample placement surface, excitation light traveling through the optical medium and having an excitation wavelength of the sample;
a probe light source to emit probe light traveling through the optical medium;
a light position detector to detect a position of emission probe light emitted from the optical medium; and
a computation circuitry to calculate an amount or a concentration of a sugar component in a biological substance, based on a position of the emission probe light detected by the light position detector when the sample is a referential substance, and a position of the emission probe light detected by the light position detector when the sample is the biological substance, the referential substance absorbing the excitation light and having no specific absorption peak in the excitation wavelength of the excitation light, wherein
the computation circuitry further calculates the amount or concentration of the sugar component, based on a position of the emission probe light detected by the light position detector when the sample is a reference substance, the reference substance absorbing the excitation light and having an absorption peak(s) of one or more sugar components in the excitation wavelength of the excitation light, and
the computation circuitry includes an inference device, and the inference device includes
a data acquisition circuitry to acquire the position of the emission probe light detected by the light position detector when the sample is the referential substance, the position of the emission probe light detected by the light position detector when the sample is the reference substance, and the position of the emission probe light detected by the light position detector when the sample is the biological substance, and
an inference circuitry to infer the amount or concentration of the sugar component, from the position of the emission probe light detected by the light position detector when the sample is the referential substance, the position of the emission probe light detected by the light position detector when the sample is the reference substance, and the position of the emission probe light detected by the light position detector when the sample is the biological substance, the positions being acquired by the data acquisition circuitry, using a learned model for inferring the amount or concentration of the sugar component from the position of the emission probe light detected by the light position detector when the sample is the referential substance, the position of the emission probe light detected by the light position detector when the sample is the reference substance, and the position of the emission probe light detected by the light position detector when the sample is the biological substance.

17. A biological component measurement device comprising:
an optical medium including a sample placement surface;
an excitation light source to emit, toward a sample placed on the sample placement surface, excitation light traveling through the optical medium and having an excitation wavelength of the sample;
a probe light source to emit probe light traveling through the optical medium;
a light position detector to detect a position of emission probe light emitted from the optical medium; and
a computation circuitry to calculate an amount or a concentration of a biological component in a biological substance, based on a position of the emission probe light detected by the light position detector when the sample is a referential substance, and a position of the emission probe light detected by the light position detector when the sample is the biological substance, the referential substance absorbing the excitation light and having no specific absorption peak in the excitation wavelength of the excitation light, wherein
the computation circuitry further calculates the amount or concentration of the biological component, based on a position of the emission probe light detected by the light position detector when the sample is a reference substance, the reference substance absorbing the excitation light and having an absorption peak(s) of one or more biological components in the excitation wavelength of the excitation light, and
the computation circuitry includes an inference device, and the inference device includes
a data acquisition circuitry to acquire the position of the emission probe light detected by the light position detector when the sample is the referential substance, the position of the emission probe light detected by the light position detector when the sample is the reference substance, and the position of the emission probe light detected by the light position detector when the sample is the biological substance,
a preprocessing circuitry to calculate one indicator, from the position of the emission probe light detected by the light position detector when the sample is the referential substance, the position of the emission probe light detected by the light position detector when the sample is the reference substance, and the position of the emission probe light detected by the light position detector when the sample is the biological substance, and
an inference circuitry to infer the amount or concentration of the biological component from the indicator calculated by the preprocessing circuitry, using a learned model for inferring the amount or concentration of the biological component from the indicator.
